(19) United States
(12) Patent Application Publication
(10) Pub. No.: US 2005/0004475 A1
(43) Pub. Date: Jan. 6, 2005

(54) Method of comparing the appearance of a region of the human body at at least two different instants

(75) Inventor: Franck Giron, Ferrieres-En-Brie (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 10/866,021

(22) Filed: Jun. 14, 2004

Related U.S. Application Data
(60) Provisional application No. 60/495,101, filed on Aug. 15, 2003.

(30) Foreign Application Priority Data
Jun. 13, 2003 (FR) 03 07172

(51) Int. Cl. A61B 6/00

(52) U.S. Cl. 600/476; 382/128; 348/77

(57) ABSTRACT

The present invention provides a method of comparing the appearance of a region of the human body at at least two different instants, the method comprising the following steps:
generating at least two 3D images (F₁, F₂) of said region, these 3D images being obtained by transforming into heights gray levels in two 2D images of said region taken respectively at said instants.

The present invention relates to a method of revealing, amongst other things, the effect of applying a substance to a region of the human body, and in particular the optical effect induced by the presence of the substance.

There exist lipsticks that enable lips to look “fuller”. There also exist nail varnishes which give a volume effect to the nails. Light/dark makeup is also often used for modeling the face.

Nevertheless, it is difficult to quantify the impression of volume that is imparted by the makeup.

The invention seeks in particular to make it easier to measure the optical effect obtained by makeup.

In a first of its aspects, the invention thus provides a method of comparing the appearance of a region of the human body at at least two different instants, the method comprising the following steps:
generating at least two 3D images of said region, these 3D images being obtained by transforming into heights gray levels of at least two 2D images of said region taken respectively at said instants.

The term “two different instants” is used in the invention to designate two times of acquisition for 2D images. These two times may differ, for example, by a few seconds or minutes, e.g. the time taken to apply a substance, in particular makeup, to the region of the body under consideration, or they may be further apart, for example several days, weeks, or months, for example when it is desired to reveal the effects of treatment by a care product, in particular an antiwrinkle product.

A “2D image” is an image in two dimensions of a region of the body. Each pixel of the 2D image is associated with two coordinates enabling it to be located in the 2D image, and with a gray level. A gray level of 0 corresponds, for example, to black in the image, and a maximum gray level, e.g. equal to 255 when encoding on 8 bits, corresponds to white, with gray levels between those two extreme values corresponding to respective intermediate shades of brightness.

The 2D image may be in color or in black and white. For a color image, the gray level may be the value of one of the tristimulus values of the signal, for example one of the red, green, and blue (R, G, B) values, or it may be a value corresponding, for example, to a mean gray level of the pixel, where such a mean gray level can be obtained, for example, by averaging the R, G, and B levels.

A “3D image” is a three-dimensional image that can be generated from a single 2D image by transforming each gray level of the 2D image into height. Each point of the 3D image then has three coordinates enabling it to be situated in space. On such a 3D image, rough relief can represent a large amount of variation in brightness, which may correspond to an increased impression of volume given by the makeup. On the contrary, relief that is substantially flat may represent makeup that is more uniform, with little variation in brightness.

The 3D image makes it possible to measure the visual impression of a decoy or camouflage effect provided by the makeup. The 3D image does not represent real relief but rather the optical illusion that is induced by the makeup. Roughness, hills, and valleys constituting the relief in the 3D image are virtual.

Topographical treatment may be performed on at least one of the 3D images.

In particular, it is possible to use software for topographical processing as though the relief in the 3D image generated by the method of the invention were real.

For example, the topographical treatment may serve to determine a roughness, or the altitude of a valley or a hill, amongst other things.

The term “image” is used to designate any set of pixels containing information about the appearance of the region of the human body under study. The 2D image may be apparent and may be viewed, e.g. by the user, in particular by means of a computer screen or a paper medium. In a variant, the 2D image need not be apparent, e.g. being in the form of a computer file. In which case, only the 3D image need be made apparent, where appropriate, in order to enable it to be compared with another 3D image. A 2D image may correspond to a fraction only of a previously acquired larger image. A 3D image may be displayed in two dimensions, in perspective or otherwise. Or indeed only a section of the 3D image need be displayed, where appropriate.

The method of the invention can be implemented using 2D images that have already been acquired, or the method may further include the step which consists in acquiring the 2D images of the region in question. The 2D images may be scanned photographs, for example.

The 2D images are preferably acquired under the same ambient conditions, and in particular under the same lighting conditions, e.g. under diffuse light.

The 2D images may be acquired by means of a sensor that is optical or not optical. An optical sensor may be constituted, for example, by an analog or a digital camera, for taking still or moving pictures. A non-optical sensor may be a sensor comprising an array of non-optical detection cells, e.g. capacitive, thermal, or other cells. The gray level of each pixel in the 2D image may represent the capacitance measured by an associated detection cell.

The method may further include the step consisting in cutting out portions of 2D images of the region of the human body under study. For example, for the lips, it is possible to select only the lips from an image of the bottom of the face. This can make it easier to compare two images, with the 3D images corresponding solely to the cutout region, e.g. the lips.

The method may also include the step of processing two 2D images in such a manner that the 2D images have the same mean gray level, before generating the corresponding 3D images. This can make the two images easier to compare.

The method advantageously includes the step consisting in enabling the 3D images that are generated to be compared, and in particular enabling variations in heights in the 3D images to be compared.

The method may include in particular the step consisting in displaying the 3D images so as to enable them to be compared visually. The 3D images may be compared simultaneously, being juxtaposed or superposed, on a screen or printed on a paper medium, for example.

The two 2D images may be acquired respectively before and after applying a substance to the region under study, for example, a makeup such as a lipstick.

An advantage of the present invention is to reveal the effect of the substance on the appearance of the region under study by comparing the 3D image of the region while uncovered with the 3D image of the region after the substance has been applied.

In a variant, both 2D images may be images of the region under study, respectively after applying first and second different substances. Under such circumstances, the method makes it possible to reveal differences in appearance depending on whether it is the first or the second substance that has been applied. For example, these first and second substances may contain one or more components of different kinds and/or at different concentrations.

The substance(s) applied may be constituted by makeup, e.g. lipstick, lip gloss, foundation makeup, or nail varnish, or by care products, e.g. substances for treating or hiding a defect of the skin, for example a wrinkle, in particular at the corner of the eye or between the eyebrows, a red mark, or a scar.

The region of the human body under study may be a region of the skin, e.g. of the face, of the mucous membranes, in particular the lips, or of hair or nails.

The method of the invention may be implemented on sales premises, in a beauty parlor, or at a person's home, for example.

It may be used to show up the effect of treatment on a skin defect, e.g. a wrinkle. The method may be performed several times so as to follow changes in the appearance of a wrinkle during treatment of long duration.

In another of its aspects, the invention also provides a method of showing up the volume-imparting effect of a lipstick or a lip gloss, the method comprising the following steps:
acquiring two 2D images of the lips respectively before and after applying lipstick or lip gloss to the lips;
generating at least two 3D images of the lips, said 3D images being obtained by transforming into heights gray levels in the two 2D images; and
enabling the 3D images to be compared, in particular the heights of the 3D images.

In another of its aspects, the invention also provides a method of showing up the visual effect of a substance applied to the skin or the nails, the method comprising the following steps:
acquiring two 2D images of the nails or of the skin respectively before and after applying the substance;
generating at least two 3D images of the lips, these 3D images being obtained by transforming into heights gray levels in the two 2D images;
enabling the 3D images to be compared, in particular the heights in the 3D images.

In another of its aspects, the invention also provides a method of showing up attenuation in the visibility of a skin defect, in particular a wrinkle, the method comprising the following steps:
acquiring two 2D images respectively before and after applying a substance to the skin defect;
generating at least two 3D images of the skin defect, these 3D images being obtained by transforming into heights gray levels of the two 2D images;
enabling the 3D images to be compared.

The substance that is applied may be a makeup or care product.

In another of its aspects, the invention also provides a method of treating a skin defect, e.g. a wrinkle, with a substance, in particular a cosmetic, the method comprising the following steps:
acquiring a 2D image of the skin defect prior to treatment;
treating the skin defect with the substance;
acquiring a 2D image of the skin defect after treatment;
enabling two 3D images of the skin defect to be compared, these 3D images being obtained by transforming into heights gray levels in the two 2D images.

In another of its aspects, the invention also provides a method enabling the appearance of two different regions of the human body to be compared, the method comprising the following steps:
acquiring a 2D image of a first region;
applying a substance to a second region;
acquiring a 2D image of the second region;
enabling two 3D images of the two regions to be compared, these two 3D images being obtained by transforming into heights gray levels of the two 2D images.

By way of example, the two regions of the human body can be two lips. For example, lipstick may be applied to one lip, e.g. the top lip, and using the above-defined method, the respective appearances of the uncovered lip, e.g. the bottom lip, can be compared with the appearance of the lip that has been made up.

In another of its aspects, the invention provides a method of comparing the appearance of a region of the human body of at least two people, the method comprising the following steps:
acquiring a 2D image of the region under consideration of each person;
enabling at least two 3D images to be compared, the 3D images being obtained by transforming into heights gray levels in the 2D images.

The method may also include a step consisting in applying a substance to the region under consideration of at least one person prior to acquiring the corresponding 2D image.

In another of its aspects, the invention provides a makeup or care kit for a region of the human body, the kit comprising:
a makeup or care product; and
at least one 3D image generated by a method as defined above, and in particular two images enabling the effect of the product to be revealed.

By way of example, the 3D image(s) may be printed on the packaging of the product or on instructions accompanying the product.

The 3D image(s) may also appear on an advertising poster, or may be broadcast by radiowaves, by satellite, by optical fiber, or by wired network, for display on a television screen, a portable terminal, or a computer.

In another of its aspects, the invention also provides a computer site, in particular an Internet site, arranged firstly to receive and store at least two 2D images of a region of the human body taken at two respective different instants and sent via a network, in particular the Internet, and also arranged to generate at least two 3D images of said region from the two 2D images, the 3D images being obtained by transforming into heights gray levels in the two 2D images.

The site may also be arranged to display the 3D images and to enable heights to be compared in the 3D images.

The site may also be arranged to perform topographical computations on the 3D images, e.g. in order to determine the roughness or the altitudes of hills or valleys.

The site may be arranged, for example, to transform variations of height in a 3D image into an index representative of the volume-imparting effect or of the attenuation of a wrinkle, for example.

The site may also be arranged to prescribe a substance for camouflaging or treating a skin defect, where appropriate.

The 2D images may be obtained using a webcam or a scanner, e.g. at the home of a user, and sent to the site which is arranged to receive them and to store them.

The site may also be arranged to cut out the 2D images so as to extract a particular region therefrom, e.g. the lips, and so as to associate each-pixel with a gray level.

In another of its aspects, the invention also provides a method of promoting product sales, in particular of cosmetics, including care products, the method including the step consisting in revealing the effectiveness of the product as observed by means of a method as defined above, or enabling at least one 3D image relating to a result obtained by the product to be viewed.

Such a promotion can be carried out using any communications channel. It can be performed, in particular, by a sales person directly at a point of sale, by radio, television, or telephone, particularly using advertising spots or short messages. It may also be carried out using press publishing or by any other document, in particular for advertising purposes. It may also be carried out over the Internet, or any other suitable computer network or mobile telephone network. It may also be carried out directly on the product, in particular on its packaging or on instructions associated therewith.

The invention will be better understood on reading the following detailed description of non-limiting implementations thereof, and on examining the accompanying drawings, in which:

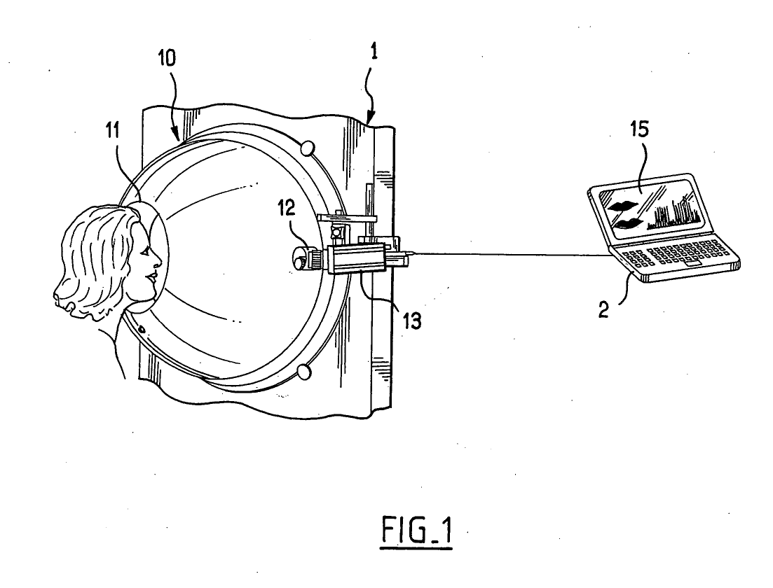

FIG. 1 is a diagram showing an example of apparatus enabling the method of the invention to be implemented;

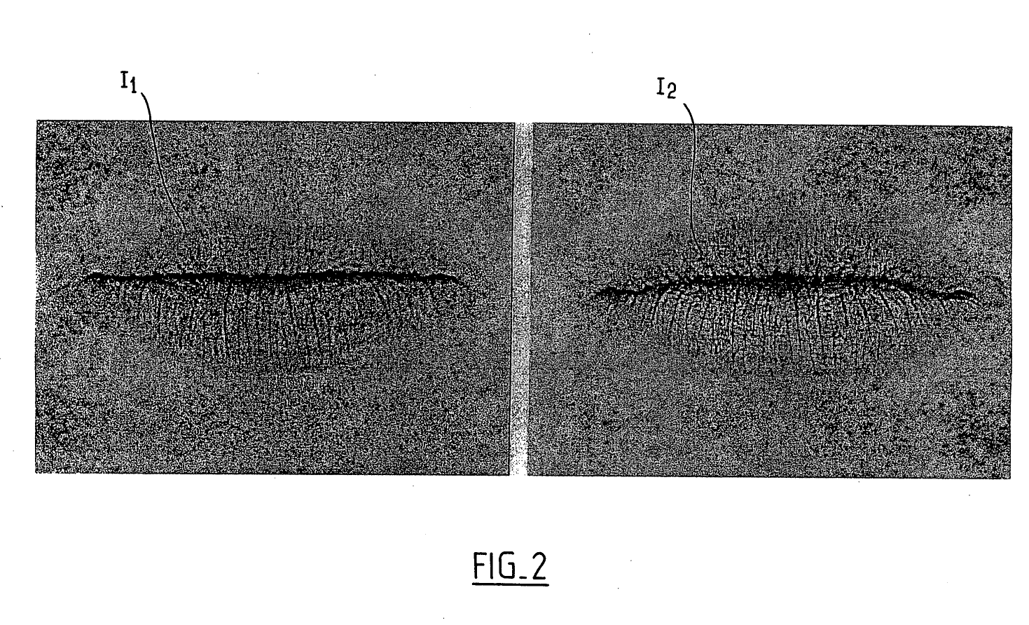

FIG. 2 shows two photographs of lips respectively without makeup and when made up;

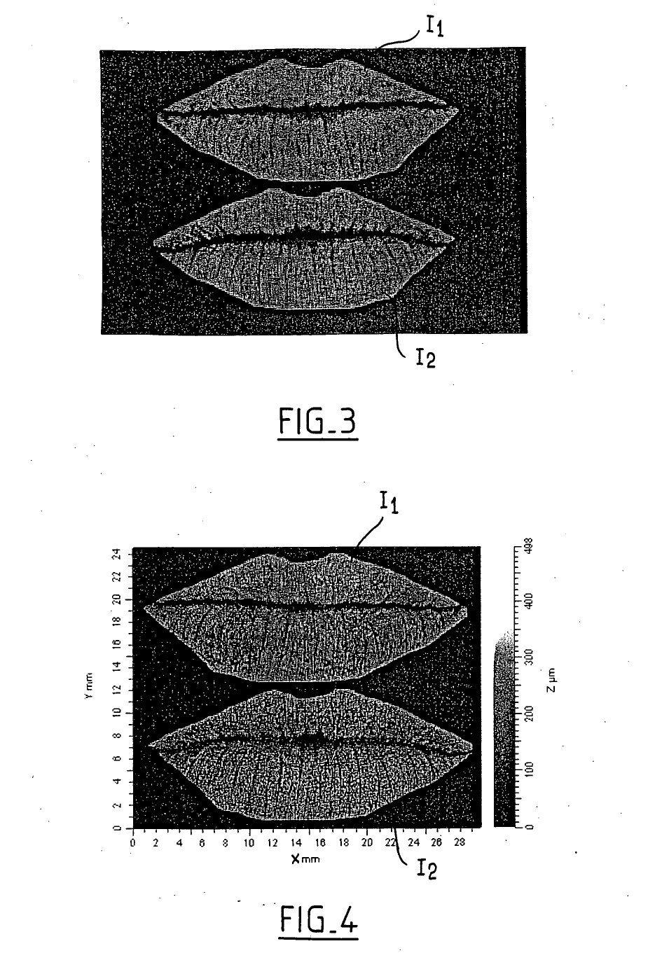

FIG. 3 shows the lips of FIG. 2 after blocking out;

FIG. 4 shows the lips of FIGS. 2 and 3 in which each pixel has been transformed into a gray level;

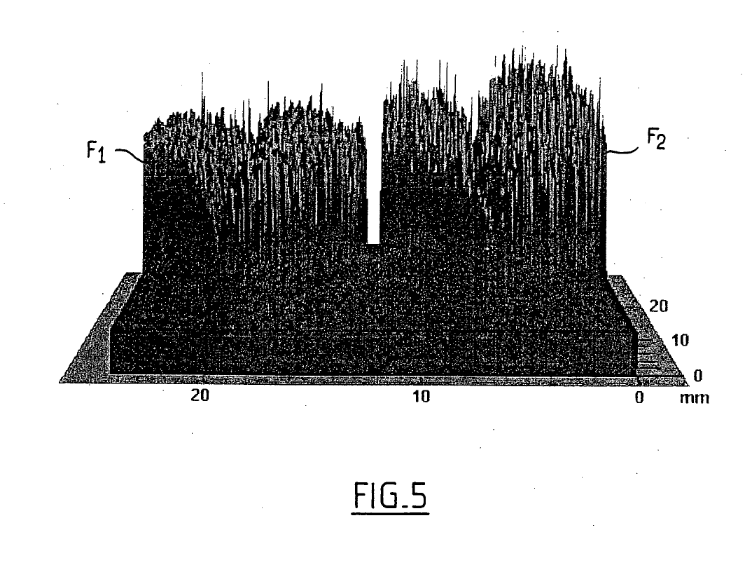

FIG. 5 shows the 3D images generated from the 2D images of FIG. 4;

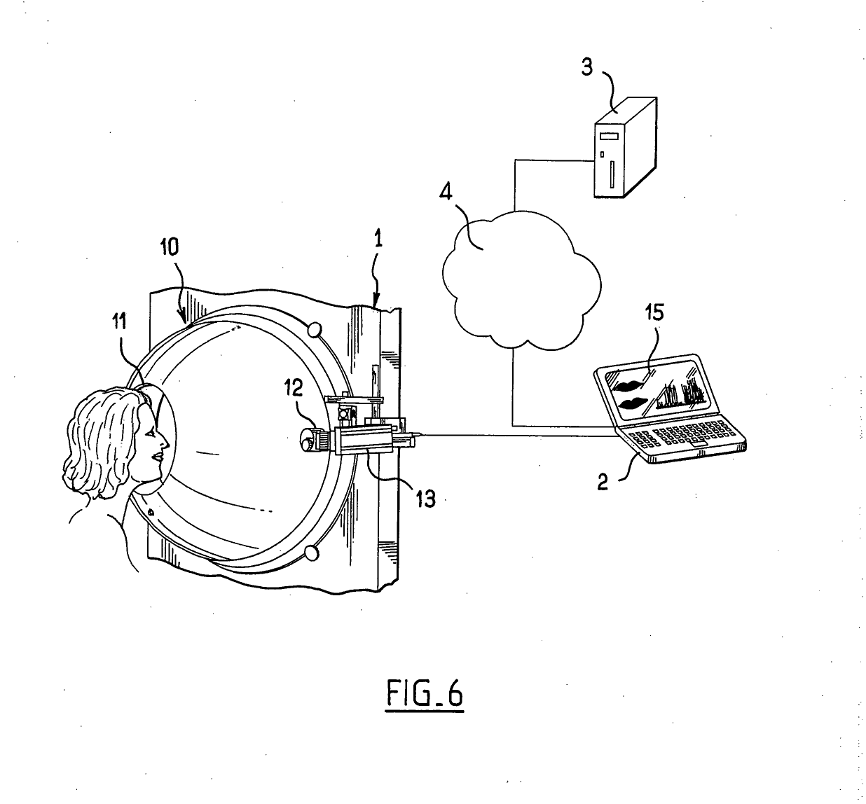

FIG. 6 is a diagram showing another example of apparatus enabling the method of the invention to be implemented;

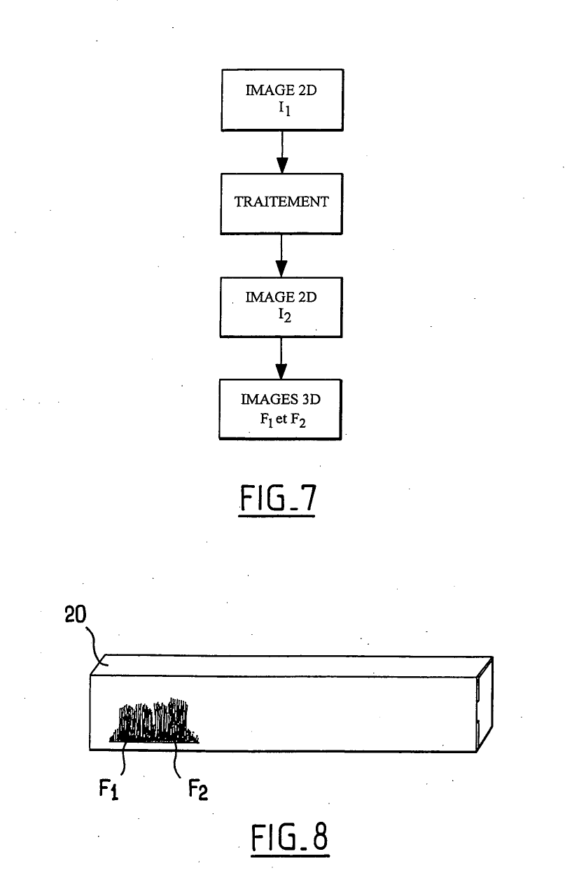

FIG. 7 is a flow chart showing a method of the invention; and

FIG. 8 is a diagrammatic and fragmentary view of a kit in accordance with the invention.

FIG. 1 shows an example of apparatus for implementing the invention. The apparatus comprises acquisition means 1 for acquiring, in the example shown, at least two 2D images of the lips of a person at two respective different instants t₁ and t₂, before and after applying lipstick.

The FIG. 1 apparatus also includes means for processing the 2D images acquired by the acquisition means 1, these processor means comprising, for example, a conventional microcomputer 2. Naturally, the microcomputer 2 is merely one example amongst a variety of processor means that could be used, it being possible for the microcomputer 2 to be replaced by a portable terminal, in particular a mobile telephone, or a personal digital assistant (PDA).

By way of example, the acquisition means 1 may comprise a substantially spherical enclosure 10 having an opening 11 enabling the user to insert the head or part of the body.

The enclosure 10 is fitted with one or more light sources which may be sources of continuous or of flashing light, which light may optionally be polarized.

Where appropriate, the light inside the enclosure 10 may be diffuse light so as to enable images to be acquired without highlights. In the example described, the enclosure 10 is provided with one or more small side or front openings 12 associated with one or more side or front cameras 13 enabling 2D images to be acquired of a portion of the person's face positioned in the opening 11 of the sphere.

Positioning means (not shown) may be arranged in the apparatus 1 so as to facilitate positioning the face in the opening 11 and enable two 2D images to be acquired under the same conditions.

Other optical acquisition means can be used, for example a webcam, a digital camera, or a scanner.

It is also possible to acquire 2D images by means of a sensor that is not optical, for example a sensor sold under the trademark TOUCHCHIP® by the supplier ST-MICROELECTRONICS. Such a sensor comprises an array of cells for detecting the capacitance of the skin.

The microcomputer 2 includes display means, in particular a screen 15, enabling each 2D image acquired by the apparatus 1 to be displayed, in particular images acquired by a single camera placed at the end of the enclosure 10 opposite from the opening 11.

The FIG. 1 apparatus can be used as follows.

A person puts their face into the opening 11, thus enabling the apparatus 1 to acquire a first image I₁ of the lips at a first instant t₁.

This two-dimensional image is stored by the microcomputer 2.

Makeup, in particular lipstick, is then applied to the lips of that person.

The face is then put back into the opening 11 and a new image I₂ of the made-up lip is acquired at a second instant t₂, and stored in the microcomputer 2.

Two examples of acquired images I₁ and I₂ are shown in FIG. 2. These images are 2D images.

The microcomputer 2 includes means enabling the lips in the acquired images I₁ and I₂ to be cut out, for example image analysis software of the OPTIMAS or PHOTOSHOP type.

FIG. 3 shows the cutout images I₁ and I₂ of the lips respectively without and with makeup.

FIG. 4 also shows a scale of gray levels.

Each pixel in each 2D image can be located on the image by an abscissa value x and an ordinate value y. Each pixel also has a gray level z which may lie in the range, for example 0 to 255. The gray levels of the images I₁ and I₂ may be adjusted so that each image has the same mean gray level.

The microcomputer 2 is arranged to transform the gray level z into height, thus enabling two so-called “3D” images F₁ and F₂ to be obtained in which each pixel has three coordinates (x, y, z), as shown in FIG. 5. The images F₁ and F₂ may be generated by TOPOSURF or OptiCAD® software, for example. In this case, the images I₁ and I₂ are initially recorded as files in a format that is readable by TOPOSURF. All or part of the resulting 3D image can be viewed, e.g. by being displayed on a screen or by being printed. In particular, it is possible to display only a section in the (z, y) or the (y, z) plane of the image, or only a fraction of the image for a range of x values or of y values.

As shown, the microcomputer 2 is preferably arranged to display the images F₁ and F₂ that it generates side by side, so as to make them easier to compare and so as to make it easier to perceive the volume-imparting effect of a lipstick, for example.

In FIG. 5, it can be seen that the relief in the left-hand image corresponding to lips that have not made up is smoother than the relief in the right-hand image corresponding to lips that have been made up. The pale zones of the lips are at higher altitude than the dark zones. The rougher the relief, the greater the contrast between the pale and dark zones, leading to a greater volume-imparting effect.

The volume-imparting effect obtained on the lips can be associated, for example, with the formulation of the lipstick, which may include, for example, at least one goniochromatic coloring agent.

The apparatus 1 and the microcomputer 2 may be present at a point of sale or in a beauty parlor.

The microcomputer 2 may be connected to a remote computer server 3 via a network 4 of the Internet or the Intranet type, for example, as shown in FIG. 6.

The microcomputer 2 can then be arranged solely to receive and store the 2D images acquired by the acquisition means 1, and to send them to the server 3 which is arranged to receive the 2D images and to process them.

The server 3 may be arranged to generate the 3D images from the 2D images and to send the 3D images generated in this way to the microcomputer 2 so as to enable the user to view the volume-imparting effect of a lipstick on the screen 15, for example.

The server 3 may be arranged to generate an index for each 3D image, said index being associated with the variations of height in the image, e.g. so as to make comparison easier.

The 3D images may be printed on a sheet, displayed on the screen 15, or where appropriate displayed in relief by any conventional means, for example comprising a liquid crystal shutter or colored glass spectacles.

The method of the invention may also be implemented to show the effect in attenuating the visibility of a skin defect, in particular a wrinkle, for example a crow's foot.

As shown in FIG. 7, the method may comprise a first step 30 consisting in acquiring an image I₁ of a defect in the region in question of the skin, for example using the acquisition means 1 of FIG. 1.

Thereafter, treatment may be performed in a second step 31, using a care product, possibly as prescribed by the microcomputer 2 or the server 3.

After or during treatment, it is possible in a third step 32 to acquire an image I₂ of the same region of the skin taken under the same conditions. The two acquired images I₁ and I₂ are 2D images, and in a step 33 they can be transformed into images F₁ and F₂ that are 3D images as described above, thus making it possible to compare the relief in the two 3D images and to show up the effect of wrinkle attenuation after the treatment. The relief of the image F₁ before treatment may be rougher than the relief of the image F₂ after treatment.

Naturally, the invention is not limited to the implementations described above. The region of the human body concerned may be any part of the face, the hair, the body, the mucous membranes, or the nails.

3D images may appear on the packaging 20 of a corresponding product, as shown in FIG. 8, where the product is constituted by lipstick or by lip gloss, for example. The two 3D images appearing on the packaging may correspond, for example, to lips that are not made up and to lips that are made up.

The product may also be a care product, and the 3D images may be associated respectively with images obtained before and after treatment.

Throughout the description, including in the claims, the term “comprising a” should be understood as being synonymous with “comprising at least one”, unless specified to the contrary.

1. A method of comparing the appearance of a region of the human body at at least two different instants (t₁, t₂), the method comprising the following steps:
generating at least two 3D images (F₁, F₂) of said region, these 3D images being obtained by transforming into heights gray levels in two 2D images (I₁, I₂) of said region taken respectively at said instants (t₁, t₂).

2. A method according to claim 1, characterized by the fact that it further includes the steps consisting in acquiring the two 2D images (I₁, I₂) of said region.

3. A method according to the preceding claim, characterized by the fact that the two 2D images (I₁, I₂) are acquired under the same ambient conditions, in particular under the same lighting conditions.

4. A method according to the preceding claim, characterized by the fact that the 2D images (I₁, I₂) are acquired under diffuse light.

5. A method according to any one of claims 2 to 4, characterized by the fact that the two 2D images (I₁, I₂) are acquired by means of a non-optical sensor.

6. A method according to any one of claims 2 to 4, characterized by the fact that the two 2D images (I₁, I₂) are acquired by means of an analog or digital camera (13).

7. A method according to any preceding claim, characterized by the fact that it further includes the step consisting in cutting out said region from each of the two 2D images (I₁, I₂).

8. A method according to any preceding claim, characterized by the fact that it includes the step consisting in processing the two 2D images (I₁, I₂) in such a manner that these 2D images have the same mean gray level.

9. A method according to any preceding claim, characterized by the fact that it further includes the step consisting in displaying the two 3D images (F₁, F₂).

10. A method according to any preceding claim, characterized by the fact that it further includes the step consisting in enabling the two 3D images (F₁, F₂) to be compared.

11. A method according to any preceding claim, characterized by the fact that the 3D images do not show real relief.

12. A method according to any preceding claim, characterized by the fact that the two 2D images (I₁, I₂) are acquired respectively before and after applying a substance to said region.

13. A method according to any one of claims 1 to 11, characterized by the fact that the two 2D images (I₁, I₂) are images of said region respectively after applying first and second different substances.

14. A method according to either one of the two immediately preceding claims, characterized by the fact that the applied substance(s) is/are makeup.

15. A method according to the preceding claim, characterized by the fact that the makeup(s) is/are lipsticks or lip gloss.

16. A method according to claim 12 or claim 13, characterized by the fact that the substances(s) applied is/are care products.

17. A method according to any preceding claim, characterized by the fact that said region is a region of skin, in particular of the face, of mucous membranes, in particular the lips, or of hair or nails.

18. A method according to any preceding claim, characterized by the fact that topographical processing is performed on at least one of the 3D images.

19. A method of showing up the volume-imparting effect of a lipstick or a lip gloss, the method being characterized by the fact that it comprises the following steps:
acquiring two 2D images (I₁, I₂) of the lips respectively before and after applying lipstick or lip gloss to the lips;
generating at least two 3D images of the lips, said 3D images being obtained by transforming into heights gray levels in the two 2D images;
enabling the 3D images (F₁, F₂) to be compared.

20. A method of showing up the visual effect of a substance applied to the skin or the nails, the method comprising the following steps:
acquiring two 2D images of the nails or of the skin respectively before and after applying the substance;
generating at least two 3D images of lips, these 3D images being obtained by transforming into heights gray levels in the two 2D images; and
enabling the 3D images to be compared, in particular the heights in the 3D images.

21. A method of showing up the attenuation in the visibility of a skin defect, in particular a wrinkle, the method comprising the following steps:
acquiring two 2D images (I₁, I₂) respectively before and after applying a substance to the skin defect;
generating at least two 3D images (F₁, F₂) of the skin defect, these 3D images being obtained by transforming into heights gray levels of the two 2D images; and
enabling the 3D images (F₁, F₂) to be compared.

22. A method of treating a skin defect, in particular a wrinkle, with a cosmetic, the method comprising the following steps:
acquiring a 2D image of the skin defect prior to treatment;
treating the skin defect with the substance;
acquiring a 2D image of the skin defect after treatment;
enabling two 3D images of the skin defect to be compared, these 3D images being obtained by transforming into heights gray levels in the two 2D images.

23. A method of comparing the appearance of two different regions of the human body, the method being characterized by the fact that it comprises the following steps:
acquiring a 2D image of a first region;
applying a substance to a second region;
acquiring a 2D image of the second region;
enabling two 3D images of the two regions to be compared, these two 3D images being obtained by transforming into heights gray levels of the two 2D images.

24. A method of comparing the appearance of a region of the human body of at least two people, the method being characterized by the fact that it comprises the following steps:
acquiring a 2D image of the region under consideration of each person; and
enabling at least two 3D images to be compared, the 3D images being obtained by transforming into heights gray levels in the 2D images.

25. A makeup or care kit for a region of the human body, the kit being characterized by the fact that it comprises:
a substance or a care product; and
at least one 3D image (F₁ and F₂) generated by a method as defined in any one of claims 1 to 18, and in particular two 3D images enabling an effect of the substance to be shown up.

26. A server hosting a computer site, in particular an Internet site, arranged to compare the appearance of a region of the human body at at least two different instants, the server comprising means:
for receiving and storing at least two 2D images of said region taken respectively at said instants and sent over a network, in particular the Internet; and
for generating at least two 3D images of said region from the two 2D images, these 3D images being obtained by transforming into heights gray levels in the two 2D images.
